(19) United States
(12) Patent Application Publication
(10) Pub. No.: US 2022/0323632 A1
(43) Pub. Date: Oct. 13, 2022

(54) BIODEGRADABLE TOBACCO-BASED ABSORBENT ARTICLE

(71) Applicants: Yuri Lee, Ellicott City, MD (US); Emily Leting Xing, Ellicott City, MD (US)

(72) Inventors: Yuri Lee, Ellicott City, MD (US); Emily Leting Xing, Ellicott City, MD (US)

(21) Appl. No.: 17/688,816

(22) Filed: Mar. 7, 2022

Related U.S. Application Data
(60) Provisional application No. 63/252,430, filed on Oct. 5, 2021.
(60) Provisional application No. 63/173,388, filed on Apr. 10, 2021.

(51) Int. Cl. A61L 15/40; A61F 13/511; A61F 13/15

(52) U.S. Cl. CPC A61L 15/40; A61F 13/51121; A61F 13/15252; A61F 2013/15943; A61F 2013/530481

(57) ABSTRACT

A biodegradable tobacco fiber and starch-based absorbent article that serves to effectively absorb bodily fluids. The tobacco-based absorbent article includes a plurality of layers: a nonwoven top sheet, an airlaid paper, an absorbent core, a laminated nonwoven back sheet, and optionally, a release paper.

### CROSS-REFERENCE TO PRIOR APPLICATIONS

This application claims priority to U.S. Provisional Application Nos. 63/173,388, filed Apr. 10, 2021 and 63/252,430, filed Oct. 5, 2021, each hereby expressly incorporated by reference in its entirety.

### FIELD

This application is directed to a biodegradable tobacco fiber- and starch-based absorbent article that serves to effectively absorb bodily fluids.

### BACKGROUND

Historically, feminine hygiene has been a taboo subject in society, and as a result, problems associated with feminine hygiene have long been neglected. This has had a detrimental effect on society and the environment, with very little mitigation. For example, over 12 billion menstrual pads are discarded annually in the U.S. A majority of these discarded pads contain over 90% plastic, and can take 800 years to decompose. The discarded pads also include chemicals that are harmful to the body and environment. For example, feminine hygiene pads can include harmful carcinogens, volatile organic compounds, phthalates, and the like, and when exposed to such chemicals, a user may develop an increased risk of cancer, asthma, and other disabilities.

Biodegradable pads, which are eco- and body-friendly were developed to address these drawbacks associated with standard menstrual pads. However, the biodegradable pads have proved to be inefficient. Typical biodegradable pads utilize cellulose pulp for their absorbent cores instead of superabsorbent polymers (SAP) that are found in standard menstrual pads. The SAP are made of more than 90% plastic that expand to retain moisture. The use of cellulose pulp in the biodegradable pads instead of SAP makes them ineffective at sufficiently absorbing bodily fluids, leaving the user in extreme discomfort because of a wet feeling, and having to change the biodegradable pads frequently. Because of these inefficiencies, users are not incentivized to use biodegradable pads.

Concurrently, the harmful use of tobacco has been prevalent for centuries, and there is a need to repurpose this crop for a more meaningful and environmentally-friendly product. Over 150 billion cigarettes are discarded annually, making it the most littered item on the planet along with being the cause for over 480,000 American deaths annually. Multiple solutions for completely eliminating the use of tobacco have been proposed. However, one of the roadblocks to eliminating tobacco use is that more than 12,000 Americans depend on tobacco cultivation to support their families, and eliminating tobacco cultivation would be economically regressive. The tobacco plant also has various advantageous properties that can assist in generating change in the world. Tobacco is a promising absorbent material for use in absorbent articles. It has a natural lignin content of 18.9%, which is lower than current popular absorbent article ingredients such as cotton. With lignin being a hydrophobic substance found in plants, less of it would make it more ideal when used for absorbency. Tobacco can also be grown without pesticides and having less contact with such substances would be preferable for a fabric for human use. The main psychoactive ingredient of tobacco—nicotine—can be extracted as it is a hydrophilic molecule that can be removed and measured through boiling and lab equipment. This application is directed to repurposing tobacco for a beneficial purpose.

### SUMMARY

The biodegradable tobacco-based absorbent article described herein is an absorbent article made primarily of tobacco fibers and tobacco starch. The absorbent article is infused with herbs, which results in a cooling effect and/or a pleasant-smelling effect for the user. The absorbent article is biodegradable, is absorbent through its superabsorbent polymer, and utilizes tobacco, tackling three major and prevalent socio-economic issues in the world today.

An exemplary embodiment is directed to a superabsorbent polymer comprising: tobacco starch, and a pore-forming source.

Another exemplary embodiment is directed to a biodegradable absorbent article comprising: a nonwoven top sheet; an airlaid paper layer; a superabsorbent polymer layer; a laminated nonwoven back sheet; and optionally, a bioplastic release paper.

Another exemplary embodiment is directed to a method of preparing a superabsorbent polymer, the method comprising: extracting tobacco starch from tobacco leaves and stalks; and adding a pore-forming source to the tobacco starch.

Another exemplary embodiment is directed to a method of forming a biodegradable absorbent article, the method comprising: providing a nonwoven top sheet; providing an airlaid paper layer; providing a superabsorbent polymer layer; providing a laminated nonwoven back sheet; and optionally, providing a bioplastic release paper.

The absorbent article absorbs a large amount of bodily fluids and decreases discomfort for the user while being eco-and-body-friendly. By using tobacco as its primary material, the absorbent article can help shift the focus from using tobacco for harmful substances like cigarettes to products that serve to help the society, economy, and environment.

### BRIEF DESCRIPTION OF DRAWINGS

These and other features of this invention will now be described with reference to the drawings of certain embodiments which are intended to illustrate and not to limit the invention.

FIG. 1 illustrates various layers of the absorbent article according to an exemplary embodiment.

FIG. 2 is a compressed view of the absorbent article illustrated in FIG. 1.

FIG. 3 illustrates various layers of an absorbent article, such as a sanitary or menstrual pad, according to an exemplary embodiment.

FIG. 4 is a compressed view of the absorbent article of FIG. 3.

### DETAILED DESCRIPTION

Further aspects, features and advantages of this invention will become apparent from the detailed description which follows.

As used herein, the singular forms “a”, “an” and “the” are intended to include the plural forms as well, unless the context clearly indicates otherwise.

In the specification and claims, the singular forms include plural referents unless the context clearly dictates otherwise. As used herein, unless specifically indicated otherwise, the word “or” is used in the “inclusive” sense of “and/or” and not the “exclusive” sense of “either/or.”

As used herein, “about” is a term of approximation and is intended to include minor variations in the literally stated amounts, as would be understood by those skilled in the art. Such variations include, for example, standard deviations associated with techniques commonly used to measure the amounts of the constituent elements or components of an inventions described herein, or other properties and characteristics. All of the values characterized by the above-described modifier “about,” are also intended to include the exact numerical values disclosed herein. Moreover, all ranges include the upper and lower limits thereof.

Any articles described herein are intended to encompass articles which consist of, consist essentially of, as well as comprise, the various constituents identified herein, unless explicitly indicated to the contrary.

As used herein, the recitation of a numerical range for a variable is intended to convey that the variable can be equal to any value(s) within that range, as well as any and all sub-ranges encompassed by the broader range. Thus, the variable can be equal to any integer value or values within the numerical range, including the end-points of the range. As an example, a variable which is described as having values between 0 and 10, can be 0, 4, 2-6, 2.75, 3.19-4.47, etc.

Unless indicated otherwise, each of the individual features or embodiments of the present specification are combinable with any other individual feature or embodiment that are described herein, without limitation. Such combinations are specifically contemplated as being within the scope of the present invention, regardless of whether they are explicitly described as a combination herein.

Technical and scientific terms used herein have the meaning commonly understood by one of skill in the art to which the present description pertains, unless otherwise defined. Reference is made herein to various methodologies and materials known to those of skill in the art.

As used herein, the term “absorbent” means capable of absorbing fluid and retaining it.

As used herein, the term “adhesive” means any substance, composition, compound and the like, that makes an object sticky when applied and/or can be used to attach one object to another object or a surface, and the like, and/or can include any suitable adhesive known in the art, including but not limited to PVA glue, water-based adhesives, and the like, but is not limited thereto.

As used herein, the term “biodegradable” means capable of being decomposed or broken down into parts, fractions or components by natural processes, most commonly through the action of microorganisms like bacteria and fungi, but are not limited thereto, and a material that can be degraded by any biological or natural method may be included.

As used herein, the term “bioplastic” means a plastic derived from natural biological substances, as opposed to petroleum. Exemplary bioplastics that can be used for the invention described herein include, but are not limited to thermoplastic starch and cellulose bioplastics.

As used herein, the term “bodily fluids” means any liquid naturally secreted by animals, including humans, including but not limited to blood, saliva, semen, discharge, mucus, urine, sweat, puss, tears, vomit, and the like, but is not limited thereof.

As used herein, the term “eco-and-body-friendly” means not harmful to the ecology, environment, society, and/or user.

As used herein, the term “nonwoven sheet” means a fabric or fabric-like material constructed from fibers interlocked through mechanical, chemical, or thermal processes or solvent treatment, but not weaving or knitting.

As used herein, the term “airlaid paper” means a nonwoven sheet whose fibers undergo the process of airlaying to form a highly elastic, soft, and drapable fabric.

As used herein, the term “laminated nonwoven back sheet” means a fabric made from the lamination of multiple nonwoven sheets incorporated as one of the outer layers in a given arrangement of multiple layers. When a single layer is used, the same can also be considered a back sheet.

As used herein, the term “naturally-occurring” means existing by the means of nature without any artificial aid or processing.

As used herein, the term “nonwoven top sheet” means a nonwoven layer or sheet used as a first layer or sheet or an outer layer or sheet of the absorbent article that is arrange to be closest to the user. When a single nonwoven layer or sheet is used, the same can also be considered a top sheet.

As used herein, the term “pesticide-free” means without any pesticide or pesticide residues, including herbicides, insecticides, fungicides, and the like, but are not limited thereof.

As used herein, the term “primary”, “primarily”, “mainly” or any variations thereof means more than 50%, i.e., indicating at least a simple majority, but may include any percentage between 50% and 100%.

As used herein, the term “release paper” means a paper or plastic whose function is to protect an adhesive layer of an article.

As used herein, the term “superabsorbent polymer” means a substance that can absorb and retain at least 100 times the amount of fluid with respect to its own mass in the fluid.

As used herein, the term “pore-forming agent” means a substance that creates pores in a material.

An exemplary embodiment is directed to a superabsorbent polymer comprising: tobacco starch, and a pore-forming source. The pore-forming source is a nitrogen source selected from urea, ammonium nitrate, or a combination thereof. The tobacco starch can be extracted from tobacco leaves and stalks.

Another exemplary embodiment is directed to an absorbent material comprising: tobacco fibers; and optionally, herbs. The absorbent material is a nonwoven sheet comprising the tobacco fibers and/or the absorbent material is an airlaid paper comprising the tobacco fibers. In another exemplary embodiment, the nonwoven sheet is infused with herbs.

Another exemplary embodiment is directed to a biodegradable absorbent article comprising: a nonwoven top sheet; an airlaid paper layer; a superabsorbent polymer layer; a laminated nonwoven back sheet; and optionally, a bioplastic release paper. The nonwoven top sheet can include tobacco fibers and/or the airlaid paper can include tobacco fibers. The biodegradable absorbent article can include an absorbent core. The absorbent core can be embedded within the airlaid paper sheet and include a superabsorbent polymer. The nonwoven top sheet can be positioned next to the absorbent core. The nonwoven top sheet can be perforated to allow bodily fluids to pass through the nonwoven top sheet.

In an exemplary embodiment, the superabsorbent polymer layer can be any polymer that can absorb and retain extremely large amounts of a liquid relative to its own mass. In an exemplary embodiment, the superabsorbent polymer can expand to hold up to 300 times its own weight.

In an exemplary embodiment, the laminated nonwoven back sheet can include a plurality of nonwoven sheets. The laminated nonwoven back sheet can also include a hydrophobic substance on an outer surface of the laminated nonwoven back sheet. In an exemplary embodiment, the hydrophobic substance can be selected from beeswax, carnauba wax, rice-bran wax, soy wax, or a combination thereof. The laminated nonwoven back sheet can also include a biodegradable adhesive on a bottom surface thereof.

In another exemplary embodiment, the bioplastic release paper can include tobacco starch and a biodegradable and flexible material. The biodegradable and flexible material can be a biopolymer, bioplastics, bioresins, and the like, but are not limited thereto.

Another exemplary embodiment is directed to a method of preparing a superabsorbent polymer, the method comprising: extracting tobacco starch from tobacco leaves and stalks; and adding a pore-forming source to the tobacco starch. The pore-forming source is a nitrogen source, which can be selected from urea, ammonium nitrate, or a combination thereof.

Another exemplary embodiment is directed to a method of forming a biodegradable absorbent article, the method comprising: providing a nonwoven top sheet; providing an airlaid paper layer; providing a superabsorbent polymer layer; providing a laminated nonwoven back sheet; and optionally, providing a bioplastic release paper. The airlaid paper can include tobacco fibers and/or the nonwoven top sheet comprises tobacco fibers.

In another exemplary embodiment, the method may further comprise: providing an absorbent core embedded in the biodegradable absorbent article, where the absorbent core comprises a superabsorbent polymer.

In another exemplary embodiment, the method may further comprise: providing an absorbent core; and embedding the absorbent core within the airlaid paper sheet, where the absorbent core comprises a superabsorbent polymer.

The absorbent article's primary ingredient is tobacco. Before use, the nicotine content of the tobacco must be completely extracted to ensure safe use of the tobacco in an absorbent article. To extract nicotine from tobacco, whole tobacco plants are placed into pots of boiling distilled water. The water is stirred every few minutes and left to simmer until two-thirds of the liquid volume is evaporated leaving only a third of the liquid. The remaining mixture is strained, and the remaining tobacco is rinsed and cleaned thoroughly with room temperature water. This boiling and cleaning process is then repeated until no nicotine is measured when the remaining mixture is tested with appropriate chromatography equipment. The nicotine extraction process described herein is not limited thereto, and any other suitable process may be employed to fully extract nicotine from tobacco. Once all the nicotine has been removed from the tobacco plant, the leaves and stalk are separated, and processed separately as described herein.

The tobacco stalks undergo a tank retting process after being submerged in tanks of water for about 24 hours to about 7 days. The tank retting process separates the tobacco fibers, which can be retrieved from the tanks, washed, and air-dried before being broken into smaller parts, pieces or components, either by hand or machinery. The broken pieces are scutched in a machine where the rubber belts grip the fiber bundles and pass them through revolving drums that beat the fibers to separate individual fibers from the tobacco stalks. The extracted fibers are then soaked in a naturally-occurring acid that naturally bleaches the fibers. The naturally-occurring acid may be organic acids, such as oxalic acid, malonic acid, succinic acid, tartaric acid, citric acid, and the like, but are not limited thereto, and any naturally-occurring acid that bleaches the extracted fibers can be used. The tobacco stalks can be processed as described herein, but is not limited thereto, and any other suitable process may be used to separate tobacco fibers from tobacco stalks.

In an exemplary embodiment, an absorbent article includes multiple layers, including but not limited to: a nonwoven top sheet, an airlaid paper, an absorbent core, a laminated nonwoven back sheet, and a release paper. The arrangement of these layers is not limited thereto, and can be such that the arrangement improves user comfort and absorbency.

The first layer of the absorbent article is the nonwoven top sheet constructed from tank-retted tobacco fibers that are fed through a carding machine's slow, sawtooth wire-covered roll to a quicker, sawtooth wire-covered main cylinder. Through this process, each individual tobacco fiber is opened and combed in the same direction. This carded fabric goes through a hydroentanglement bonding process, where multiple high-velocity and high-pressure water jets are directed at the carded fabric. The intensity of the water jets and the pattern of the supporting drum causes the fibers to spin around one another and create a soft, yet extremely strong nonwoven sheet. This sheet is then optionally dip-dried in an eco-and-body-friendly herb mixture The herb mixture can include aloe vera, mint, and the like, but are not limited thereto, and any herb or herbaceous product suitable for use can be used. The aloe vera in aloe vera gel and menthol in mint extract have been proven to help regulate hormones during menstruation, balance pH, reduce odor, inflammation, and any nonessential bacteria.

In an exemplary embodiment, the nonwoven top sheet may have a width of about 55 mm to about 300 mm, about 60 mm to about 250 mm, about 70 mm to about 200 mm, about 65 mm to about 150 mm, about 70 mm to about 100 mm, and the like. The width of the nonwoven top sheet can be equal to any integer value or values within any of the above-described numerical ranges, including the end-points of these ranges.

In an exemplary embodiment, the nonwoven top sheet may have a length of about 120 mm to about 600 mm, about 140 mm to about 550 mm, about 160 mm to about 500 mm, about 180 mm to about 450 mm, about 200 mm to about 400 mm, about 220 mm to about 350 mm, about 240 mm to about 300 mm, and the like. The length of the nonwoven top sheet can be equal to any integer value or values within any of the above-described numerical ranges, including the end-points of these ranges.

In an exemplary embodiment, the nonwoven top sheet may have a thickness of about 0.2 mm to about 1.5 mm, about 0.3 mm to about 1.3 mm, about 0.4 mm to about 1.1 mm, about 0.5 mm to about 0.9 mm, about 0.6 mm to about 0.7 mm, and the like. The thickness of the nonwoven top sheet can be equal to any integer value or values within any of the above-described numerical ranges, including the end-points of these ranges.

The airlaid paper is devised from the same process as the nonwoven top sheet, except that the tank-retted tobacco fibers undergo an airlaying process as opposed to carding. Air is pulled through the fibers onto a drum with negative pressure that collects the fibers as a web, which are put through a hydroentanglement bonding process. During the hydroentanglement bonding process, multiple high-velocity and high-pressure water jets are directed at the carded fabric. The intensity of the water jets and the pattern of the supporting drum causes the fibers to spin around one another and create a soft, yet extremely strong airlaid paper comprising a nonwoven sheet. The airlaid paper may be prepared using the process described herein, but is not limited thereto, and any suitable process that results in the formation of a tobacco-based airlaid paper may be used. The resulting product is a durable airlaid paper that absorbs fluids from the top sheet of the absorbent article to prevent any pooling of fluids.

In an exemplary embodiment, the airlaid paper may have a width of about 55 mm to about 300 mm, about 60 mm to about 250 mm, about 70 mm to about 200 mm, about 65 mm to about 150 mm, about 70 mm to about 100 mm, and the like. The width of the airlaid paper can be equal to any integer value or values within any of the above-described numerical ranges, including the end-points of these ranges.

In an exemplary embodiment, the airlaid paper may have a length of about 120 mm to about 600 mm, about 140 mm to about 550 mm, about 160 mm to about 500 mm, about 180 mm to about 450 mm, about 200 mm to about 400 mm, about 220 mm to about 350 mm, about 240 mm to about 300 mm, and the like. The length of the airlaid paper can be equal to any integer value or values within any of the above-described numerical ranges, including the end-points of these ranges.

In an exemplary embodiment, the airlaid paper may have a thickness of about 0.2 mm to about 1.5 mm, about 0.3 mm to about 1.3 mm, about 0.4 mm to about 1.1 mm, about 0.5 mm to about 0.9 mm, about 0.6 mm to about 0.7 mm, and the like. The thickness of the airlaid paper can be equal to any integer value or values within any of the above-described numerical ranges, including the end-points of these ranges.

The absorbent core includes biodegradable tobacco-starch-based super absorbent polymer (SAP) embedded within an airlaid paper sheet. To make the SAP, tobacco starch is extracted from its leaves by soaking the tobacco leaves in a room temperature alkaline solution for about 24 hours to about 7 days and then strained. Any suitable alkaline solution can be used in this process, including but not limited to, ammonia, ammonium hydroxide, calcium hydroxide, calcium oxide, potassium hydroxide, potassium carbonate, sodium hydroxide, sodium carbonate, hydroxides, peroxides, silicates, or combinations thereof. After straining, the remaining solid is rinsed several times in an alkaline and distilled water solution at room temperature. Next, the starch is extracted from the residual rinsing water using a centrifuge machine where a central rotor spins the residual rinsing water, causing starch to sediment. The tobacco starch is collected, washed with water, and air-dried. The collected tobacco starch is then dissolved in an alkaline solution. Any suitable alkaline solution can be used in this process, including but not limited to, ammonia, ammonium hydroxide, calcium hydroxide, calcium oxide, potassium hydroxide, potassium carbonate, sodium hydroxide, sodium carbonate, hydroxides, peroxides, silicates, or combinations thereof. A nitrogen source and pore-performing agent, such as urea and/or ammonium nitrate, is added to the mixture which increases the SAPs strength and absorbency. The resulting gel-like polymer is synthesized to create a superabsorbent substance that is uniformly sprinkled onto an airlaid paper until at least 50% of the paper is in contact with the SAP.

In an exemplary embodiment, the absorbent core may have a width of about 55 mm to about 300 mm, about 60 mm to about 250 mm, about 70 mm to about 200 mm, about 65 mm to about 150 mm, about 70 mm to about 100 mm, and the like. The width of the absorbent core can be equal to any integer value or values within any of the above-described numerical ranges, including the end-points of these ranges.

In an exemplary embodiment, the absorbent core may have a length of about 120 mm to about 600 mm, about 140 mm to about 550 mm, about 160 mm to about 500 mm, about 180 mm to about 450 mm, about 200 mm to about 400 mm, about 220 mm to about 350 mm, about 240 mm to about 300 mm, and the like. The length of the absorbent core can be equal to any integer value or values within any of the above-described numerical ranges, including the end-points of these ranges.

In an exemplary embodiment, the absorbent core may have a thickness of about 0.2 mm to about 1.5 mm, about 0.3 mm to about 1.3 mm, about 0.4 mm to about 1.1 mm, about 0.5 mm to about 0.9 mm, about 0.6 mm to about 0.7 mm, and the like. The thickness of the absorbent core can be equal to any integer value or values within any of the above-described numerical ranges, including the end-points of these ranges.

The laminated nonwoven back sheet is made of two to ten previously-described tobacco-based nonwoven sheets. A plurality of nonwoven sheets can be fed into a lamination machine. An exemplary lamination method includes using an ultrasonic lamination machine. Acoustic energy is generated through a horn which outputs tuned pulses to act as a pressure wave hammer. The high speeds of the vibrations generate heat and bond the materials together, completing the laminated nonwoven back sheet. The back sheet is dip-dried in a biodegradable and hydrophobic substance, such as beeswax, rice-bran wax, carnauba wax, soy wax, and the like, and any suitable biodegradable and hydrophobic substance can be used. A biodegradable adhesive can be added to a bottom surface of the absorbent article so that the absorbent article can be attached to a material, fabric, garment or undergarment. The back sheet prevents the leakage of bodily fluids and shifting of the absorbent article when in use, ensuring the user's comfort.

In an exemplary embodiment, the laminated nonwoven back sheet may have a width of about 55 mm to about 300 mm, about 60 mm to about 250 mm, about 70 mm to about 200 mm, about 65 mm to about 150 mm, about 70 mm to about 100 mm, and the like. The width of the laminated nonwoven back sheet can be equal to any integer value or values within any of the above-described numerical ranges, including the end-points of these ranges.

In an exemplary embodiment, the laminated nonwoven back sheet may have a length of about 120 mm to about 600 mm, about 140 mm to about 550 mm, about 160 mm to about 500 mm, about 180 mm to about 450 mm, about 200 mm to about 400 mm, about 220 mm to about 350 mm, about 240 mm to about 300 mm, and the like. The length of the laminated nonwoven back sheet can be equal to any integer value or values within any of the above-described numerical ranges, including the end-points of these ranges.

In an exemplary embodiment, the laminated nonwoven back sheet may have a thickness of about 0.2 mm to about 1.5 mm, about 0.3 mm to about 1.3 mm, about 0.4 mm to about 1.1 mm, about 0.5 mm to about 0.9 mm, about 0.6 mm to about 0.7 mm, and the like. The thickness of the laminated nonwoven back sheet can be equal to any integer value or values within any of the above-described numerical ranges, including the end-points of these ranges.

A release paper comprising a tobacco starch-based bioplastic can also be included. Isolated tobacco starch is added to a mixture of starch, glycerin, and water. The mixture is heated on a hot plate and stirred until thick and transparent. The starch mixture is thinly poured onto a mold, with the resulting product being a tobacco starch-based bioplastic. Any alternate methods for forming the tobacco starch-based bioplastic layer can also be used. The tobacco bioplastic protects the absorbent article's adhesive until use, keeping it unexposed to ambient atmosphere and the elements.

In an exemplary embodiment, the release paper may have a width of about 55 mm to about 300 mm, about 60 mm to about 250 mm, about 70 mm to about 200 mm, about 65 mm to about 150 mm, about 70 mm to about 100 mm, and the like. The width of the release paper can be equal to any integer value or values within any of the above-described numerical ranges, including the end-points of these ranges.

In an exemplary embodiment, the release paper may have a length of about 120 mm to about 600 mm, about 140 mm to about 550 mm, about 160 mm to about 500 mm, about 180 mm to about 450 mm, about 200 mm to about 400 mm, about 220 mm to about 350 mm, about 240 mm to about 300 mm, and the like. The length of the release paper can be equal to any integer value or values within any of the above-described numerical ranges, including the end-points of these ranges.

In an exemplary embodiment, the release paper may have a thickness of about 0.2 mm to about 1.5 mm, about 0.3 mm to about 1.3 mm, about 0.4 mm to about 1.1 mm, about 0.5 mm to about 0.9 mm, about 0.6 mm to about 0.7 mm, and the like. The thickness of the release paper can be equal to any integer value or values within any of the above-described numerical ranges, including the end-points of these ranges.

The various layers described herein may be of any suitable length, width and thickness to enable their use in an absorbent article.

In an exemplary embodiment, the absorbent article may have a width of about 55 mm to about 300 mm, about 60 mm to about 250 mm, about 70 mm to about 200 mm, about 65 mm to about 150 mm, about 70 mm to about 100 mm, and the like. The width of the absorbent article can be equal to any integer value or values within any of the above-described numerical ranges, including the end-points of these ranges.

In an exemplary embodiment, the absorbent article may have a length of about 120 mm to about 600 mm, about 140 mm to about 550 mm, about 160 mm to about 500 mm, about 180 mm to about 450 mm, about 200 mm to about 400 mm, about 220 mm to about 350 mm, about 240 mm to about 300 mm, and the like. The length of the absorbent article can be equal to any integer value or values within any of the above-described numerical ranges, including the end-points of these ranges.

In an exemplary embodiment, the absorbent article may have a thickness of about 2 mm to about 50 mm, about 5 mm to about 45 mm, about 10 mm to about 40 mm, about 15 mm to about 35 mm, about 20 mm to about 30 mm, and the like. The thickness of the absorbent article can be equal to any integer value or values within any of the above-described numerical ranges, including the end-points of these ranges.

The absorbent article can be incorporated into numerous products, including but not limited to, sanitary pads of different sizes and absorbency levels, diapers, surgical masks, incontinence products, and/or embodied into another absorbent article, and/or incorporated into another absorbent article.

If incorporated in a menstrual pad, the menstrual pad may optionally include extension portions protruding from the sides of the pad (“wings”). The extension portions can be of any suitable shape including a square, a rectangle, a semi-circular shape, a circular shape, an ellipsoidal shape, an oval shape, and the like, or any combination thereof. Such extension portions are used to additionally secure the absorbent articles on material, fiber, garments, or undergarments, provide additional absorbency and/or protect any underlying material, fiber, garments, or undergarments from leakage.

In an exemplary embodiment, where the absorbent articles includes an extension portion, the width of the absorbent article may be about 60 to from about 190 mm, about 70 mm to from about 170 mm, about 80 mm to from about 150 mm, about 90 mm to from about 130 mm, 100 mm to from about 110 mm, and the like. The width of the absorbent article can be equal to any integer value or values within any of the above-described numerical ranges, including the end-points of these ranges.

In an exemplary embodiment, where the absorbent articles includes an extension portion, the length of the absorbent article may be about 120 mm to about 350 mm, about 140 mm to about 320 mm, about 150 mm to about 290 mm, about 160 mm to about 260 mm, about 170 mm to about 230 mm, and the like. The length of the absorbent article can be equal to any integer value or values within any of the above-described numerical ranges, including the end-points of these ranges.

In an exemplary embodiment, where the absorbent articles includes an extension portion, the thickness of the absorbent article may be about 2 mm to about 15 mm, about 3 mm to about 13 mm, about 4 mm to about 11 mm, about 5 mm to about 9 mm, about 6 mm to about 7 mm, and the like. The thickness of the absorbent article can be equal to any integer value or values within any of the above-described numerical ranges, including the end-points of these ranges.

In an exemplary embodiment, the absorbent article can be absorb bodily fluids for about 2 hours to about 24 hours, about 3 hours to about 20 hours, about 4 hours to about 16 hours, about 6 hours to about 12 hours, about 8 hours to about 10 hours, and the like. The time of absorption can be equal to any integer value or values within any of the above-described numerical ranges, including the end-points of these ranges.

In another exemplary embodiment, the absorbent article can include about 50,000 mm³ to about 300,000 mm³, about 100,000 mm³ to about 250,000 mm³, about 125,000 mm³ to about 200,000 mm³, about 187,200 mm³, and the like, of tobacco fibers. The amount of tobacco fibers can be equal to any integer value or values within any of the above-described numerical ranges, including the end-points of these ranges.

In another exemplary embodiment, the absorbent article can include 10,000 mm³ to about 100,000 mm³, about 15,000 mm³ to about 85,000 mm³, about 20,000 mm³ to about 200,000 mm³, about 43,414 mm³ of tobacco starch. The amount of tobacco starch can be equal to any integer value or values within any of the above-described numerical ranges, including the end-points of these ranges.

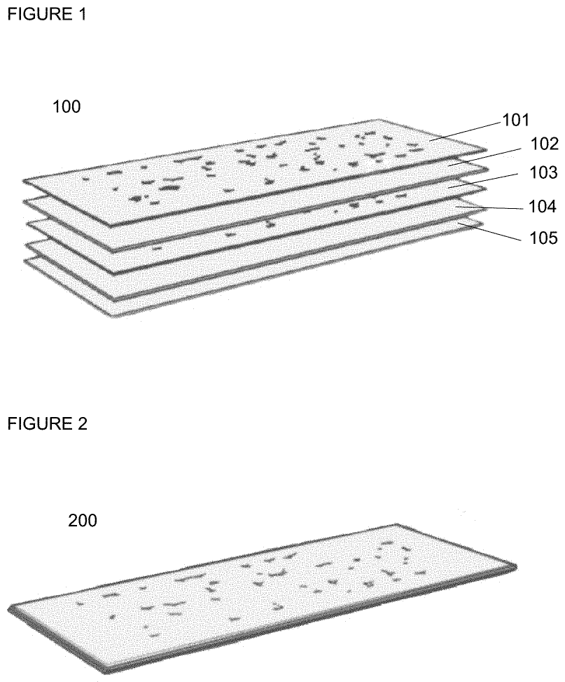

Exemplary embodiments of this application are illustrated in FIGS. 1 to 4.

An absorbent article 100 according to an exemplary embodiment is illustrated in FIG. 1. As illustrated in FIG. 1, the absorbent article 100 includes a nonwoven top sheet 101, an airlaid paper layer 102, an absorbent core 103, a laminated nonwoven back sheet 104 and a release paper 105. FIG. 2 is a compressed view 200 of the absorbent article 100 of FIG. 1.

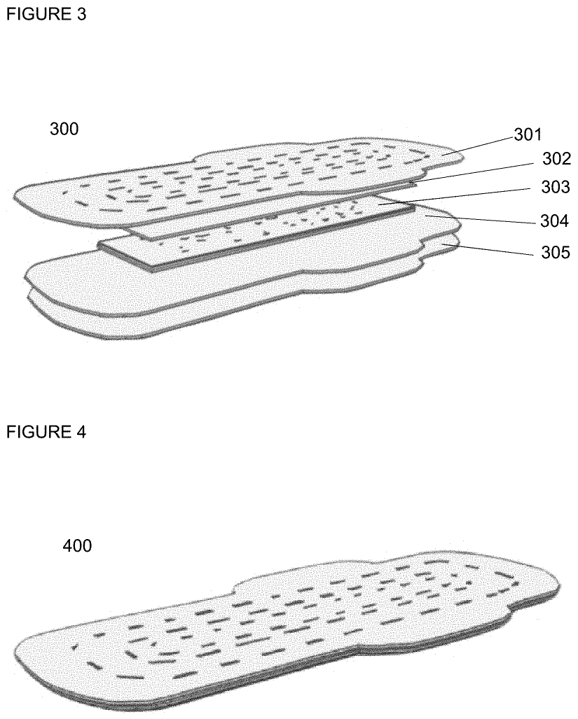

An absorbent article 300 according to another exemplary embodiment is illustrated in FIG. 3. As illustrated in FIG. 3, the absorbent article 300, which may be a menstrual pad, includes a nonwoven top sheet 301, an airlaid paper layer 302, an absorbent core 303, a laminated nonwoven back sheet 304, and a release paper 305. FIG. 4 is a compressed view 400 of the absorbent article 300 of FIG. 3.

As various changes could be made in the above methods and compositions without departing from the scope of the invention, it is intended that all matter contained in the above description shall be interpreted as illustrative and not in a limiting sense. Any numbers expressing quantities of ingredients, constituents, reaction conditions, and so forth used in the specification are to be interpreted as encompassing the exact numerical values identified herein, as well as being modified in all instances by the term “about.” Notwithstanding that the numerical ranges and parameters setting forth, the broad scope of the subject matter presented herein are approximations, the numerical values set forth are indicated as precisely as possible. Any numerical value, however, may inherently contain certain errors or inaccuracies as evident from the standard deviation found in their respective measurement techniques. None of the features recited herein should be interpreted as invoking 35 U.S.C. § 112, paragraph 6, unless the term “means” is explicitly used.

What is claimed is:

1. A biodegradable absorbent article comprising: a nonwoven top sheet; an airlaid paper layer; a superabsorbent polymer layer; a laminated nonwoven back sheet; and optionally, a bioplastic release paper.

2. The biodegradable absorbent article of claim 1, wherein the nonwoven top sheet comprises tobacco fibers.

3. The biodegradable absorbent article of claim 1, wherein the airlaid paper comprises tobacco fibers.

4. The biodegradable absorbent article of claim 1, wherein the biodegradable absorbent article further comprises an absorbent core.

5. The biodegradable absorbent article of claim 4, wherein the absorbent core is embedded within the airlaid paper sheet and comprises a superabsorbent polymer.

6. The biodegradable absorbent article of claim 1, wherein the nonwoven top sheet is positioned next to the absorbent core.

7. The biodegradable absorbent article of claim 1, wherein the nonwoven top sheet is perforated to allow bodily fluids to pass through the nonwoven top sheet.

8. The biodegradable absorbent article of claim 1, wherein the superabsorbent polymer layer can expand to hold up to 300 times its own weight.

9. The biodegradable absorbent article of claim 1, wherein the laminated nonwoven back sheet comprises a plurality of nonwoven sheets.

10. The biodegradable absorbent article of claim 1, wherein the laminated nonwoven back sheet comprises a hydrophobic substance on an outer surface of the laminated nonwoven back sheet.

11. The biodegradable absorbent article of claim 10, wherein the hydrophobic substance is selected from beeswax, carnauba wax, rice-bran wax, soy wax, or a combination thereof.

12. The biodegradable absorbent article of claim 1, wherein the laminated nonwoven back sheet has a biodegradable adhesive on a bottom surface thereof.

13. The biodegradable absorbent article of claim 1, wherein the biodegradable absorbent article comprises the bioplastic release paper, and the bioplastic release paper comprises tobacco starch and a biodegradable and flexible material.

14. The biodegradable absorbent article of claim 13, wherein the biodegradable and flexible material is selected from biopolymer, biplastics, bioresins, or a combination thereof.

15. A method of forming a biodegradable absorbent article, the method comprising:
providing a nonwoven top sheet;
providing an airlaid paper layer;
providing a superabsorbent polymer layer;
providing a laminated nonwoven back sheet; and
optionally, providing a bioplastic release paper.

16. The method of claim 15, wherein the airlaid paper comprises tobacco fibers.

17. The method of claim 15, wherein the nonwoven top sheet comprises tobacco fibers.

18. The method of claim 15, further comprising:
providing an absorbent core embedded in the biodegradable absorbent article,
wherein the absorbent core comprises a superabsorbent polymer.

19. The method of claim 15, further comprising:
providing an absorbent core; and
embedding the absorbent core within the airlaid paper sheet,
wherein the absorbent core comprises a superabsorbent polymer.
